(19) United States
(12) Patent Application Publication
(10) Pub. No.: US 2020/0315746 A1
(43) Pub. Date: Oct. 8, 2020

(54) ORTHODONTIC ALIGNER, ASSEMBLY AND METHOD FOR MANUFACTURING THEREOF

(71) Applicant: Wei-Cheng CHANG, Taichung City (TW)

(72) Inventor: Wei-Cheng CHANG, Taichung City (TW)

(21) Appl. No.: 16/843,210

(22) Filed: Apr. 8, 2020

(30) Foreign Application Priority Data
Apr. 8, 2019 (TW) 108112123
Apr. 8, 2019 (TW) 108204211
Mar. 20, 2020 (TW) 109109518

(51) Int. Cl. A61C 7/08; A61C 7/00; G16H 30/40; G16H 50/50

(52) U.S. Cl. CPC A61C 7/08; A61C 7/002; A61C 2007/004; G16H 50/50; G16H 30/40

(57) ABSTRACT

This present invention provides an orthodontic aligner installed on a user's teeth which includes a target tooth and an anchor tooth, the orthodontic aligner comprising a shell having a space constructed by receding of the shell, and a recess formed by recessing from one side of edge of the shell, and an extension formed by protruding from another side of edge of the shell, wherein the recess is located corresponding to position of the target tooth and the extension portion is located corresponding to position of the anchor tooth. This present invention also provides an orthodontic aligner assembly and manufacturing method therefor. So, only the target tooth needs to be moved will be moved during orthodontic treatment processes which can reduce sequels and the target tooth could make a predictable and long distance movement because of the recess without changing aligner frequently.

### BACKGROUND OF THE INVENTION

### Field of the Invention

The present invention relates to a medical device, more particularly to an orthodontic aligner, assembly and method for manufacturing thereof.

### Description of the Prior Art

The removal of teeth are required for many medical procedures such as tooth decay, cracked teeth or periodontitis, or insufficient spaces on gums, misaligned teeth, and other considerations for beauty. Other teeth need to be aligned or orthodontic treatment to close the space created after a tooth has been removed. Since beauty is very important for modern people, many people choose clear aligners as a major tool for orthodontic treatment to align their teeth.

However, currently each set of clear aligners can only move each tooth about 0.3 to 0.4 millimeters. Thus, each user would require about 50 to 100 sets to complete the treatment, which is very costly and wastes materials. Furthermore, during the orthodontic treatment process, some teeth don't need to be moved, causing unnecessary rotation or tilting.

### SUMMARY OF THE INVENTION

To solve the above defects, one purpose of this present invention is to provide an orthodontic aligner and assembly, and manufacturing method thereof utilized to complete the orthodontic treatment in fewer sets and minimizes the side effects such as tilting and rotation that occur.

To achieve the above objective, the present invention provides an orthodontic aligner placed on a user's teeth which include a target tooth and an anchor tooth. The orthodontic aligner comprises a shell having a space constructed by the receding of the shell, and a recess constructed through recessing of an edge of the shell at the location corresponding to the target tooth, and an extension portion protruding from the same side of the edge of the shell at the location of the anchor tooth.

To achieve the above objective, the present invention provides an orthodontic aligner assembly comprising said orthodontic aligner, a connector and an elastic member. The connector has an attaching portion and a hitching portion protruding from the attaching portion and the elastic member with two ends connect respectively to the fixing lump and the hitching portion.

To achieve the above objective, the present invention provides a manufacturing method comprising following steps: (a) taking a three-dimensional image of user's teeth before orthodontic treatment; (b) simulating a three-dimensional image of user's teeth after orthodontic treatment; (c) drawing a route map of the target teeth by comparing the three-dimensional images of user's teeth before and after orthodontic treatment; and (d) simulating the moving track and the position and size of the recess and setting it based on the route map.

According to the structures and technical features, the orthodontic aligner, assembly, and manufacturing method thereof disclosed in this invention reduces the sets needed to complete an orthodontic treatment, lowers costs and minimizes the waste of materials. Only the target tooth will be moved and the anchor teeth will stay stationary, thereby eliminating the need for an invasive temporary anchorage device.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a breakdown drawing to illustrate an embodiment of this present invention of an orthodontic aligner and assembly.

FIG. 2 is a schematic drawing to illustrate an embodiment of this present invention of an orthodontic aligner and assembly with a button as the connector when it is in use.

FIG. 3 is a schematic drawing for close-up of the recess and the connector to illustrate an embodiment of this present invention.

FIG. 4 is a schematic drawing to illustrate another embodiment of this present invention of an orthodontic aligner and assembly with a hook as the connector when it is in use.

FIG. 5 is a flow chart for manufacturing method of an orthodontic aligner of this present invention.

FIG. 6 is a three-dimensional image of user's teeth before orthodontic treatment.

FIG. 7 is a route map of the target teeth.

FIG. 8 is a three-dimensional image of user's teeth after orthodontic treatment.

FIG. 9 is an overlapping comparison of the three-dimensional images of user's teeth before and after orthodontic treatment.

### DETAILED DESCRIPTION OF THE EMBODIMENT OF THE INVENTION

The specific embodiment described below is going to illustrate a practical example of this present invention, but not to limit the protection scope of this present invention.

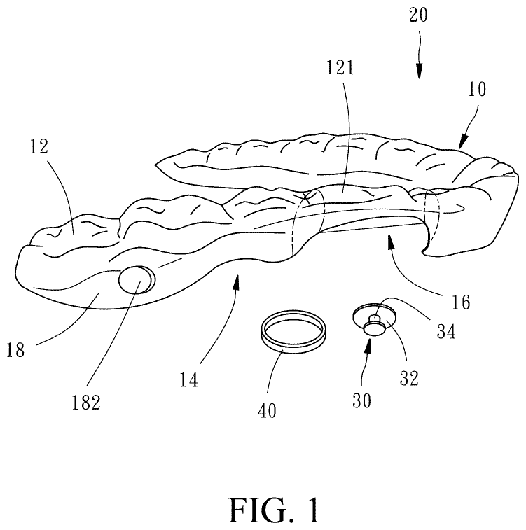

FIGS. 1˜2 illustrate an embodiment of an orthodontic aligner 10 and an orthodontic assembly 20 including the orthodontic aligner 10. Wherein, the orthodontic aligner 10 is placed on a user's teeth which includes a target tooth 101 and at least one anchor tooth 102. The target tooth 101 is a user's tooth which will be moved during this orthodontic treatment. The anchor tooth 102 is one or multiple teeth which provide a stationary point and fixing force. In order to describe the technical features of the orthodontic aligner 10 and the orthodontic assembly 20, the top and bottom from the perspective of FIG. 1 are defined as the top and bottom of the orthodontic aligner 10 and the orthodontic assembly 20, and the orthodontic aligner 10 and the orthodontic assembly 20 are placed on user's lower teeth in this embodiment. It is therefore understandable that the top and bottom of the orthodontic aligner 10 and the orthodontic assembly 20 should be reversed when they are placed on user's upper teeth.

The orthodontic aligner 10 includes a shell 12, a space 14 constructed by receding of the shell 12, a recess 16 constructed through upwardly recessing from an edge of the shell 12 at the location corresponding to the target tooth 101, and an extension portion 18 downwardly protruding from the same side of the edge of the shell 12 at the location corresponding to the anchor tooth 102. The shell 12 is set to cover a user's lower teeth to put the lower teeth into the space 14. The extension portion 18 further protrudes a fixing lump 182.

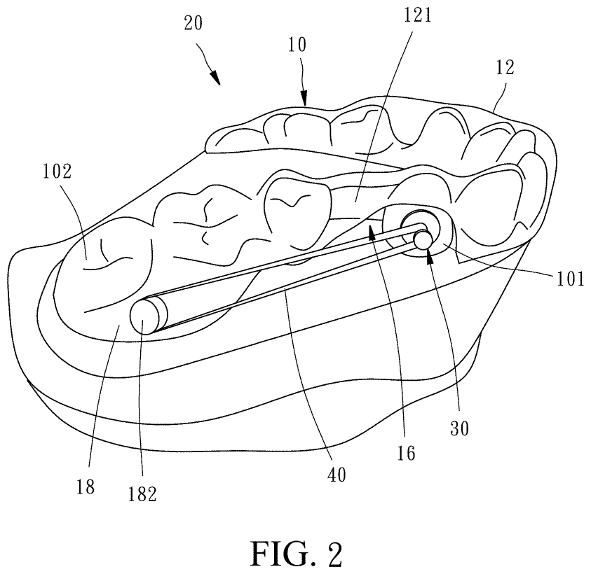

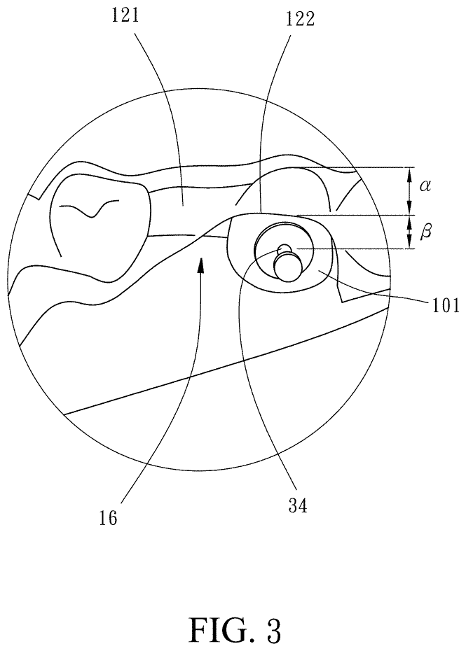

As FIG. 1 shows, the shell 12 has a wall 121 where the recess 16 formed. One of sections, the area circumscribed by broken lines in the FIG. 1, the section around the wall 121 forms a moving track 122. A side of the moving track 122 is the wall 121. As illustrated in FIGS. 2 and 3, a vertical distance a between the wall 121 of the shell 12 and the top of crown of the target tooth 101 is about 2 to 4 mm which makes the shell 12 partially covers the top of crown of the target tooth 101 and reveals the outer side of it behind the recess 16. The fixing lump 182 is located corresponding to the center of resistance of the anchor tooth 102 where the extension portion 18 locates correspondingly. Wherein, the center of resistance is an important concept in orthodontic means the quality center of a tooth. A tooth will only move vertically without rotation when the forces pass through the center of resistance during orthodontic treatment. So, the fixing lump 182 can provide forces to fix and anchor by whole structure of the shell 12 covering on user's teeth and the anchor tooth 102 correspondingly to achieve a steady effect. Furthermore, the center of resistance will locate on the extension portion line linked by points of the center of resistance of these teeth if the anchor teeth 102 are multiple teeth.

In order to facilitate the process of orthodontic treatment, locations of the recess 16, the extension portion 18 and the fixing lump 182 are better the same outer side of user's teeth but not limited of it. Wherein, the shell 12 is made of clear and hard materials including but not limited to PET, PET-G, TPU-PET-G, PE, EVA, PC or PVC. The thickness of the shell 12 is between 0.25 to 0.6 inches, about 0.625 to 1.5 mm to make it harder and/or steadier.

Further illustrated in FIGS. 1 to 3, the orthodontic aligner assembly 20 includes the aligner 10, a connector 30 and an elastic member 40. The connector 30 has an attaching portion 32 and a hitching portion 34 protruding from the attaching portion 32 which attach on the outer side of the crown of the target tooth 101. The connector 30 can be common dental instruments in practice including but not limited to a bottom. Two ends of the elastic member 40 connect respectively to the fixing lump 182 and the hitching portion 34. A tensile force of the elastic member 40 pull the target tooth 101 towards the fixing lump 182. In this embodiment, the elastic member 40 is a rubber band wrapped around on the fixing lump 182 and the hitching portion 34. In other possible embodiment, the elastic member 40 can be other elements with the same effect. The vertical distance β between the wall 121 and the central position of the hitching portion 34 is about 2 mm which makes it convenient to attach the connector 30 to the crown of the target tooth 101 and proceeding orthodontic treatment like attaching on the elastic member 40.

In this embodiment, the target tooth 101 is a lower canine at the right side, the anchor tooth 102 is a lower molar at the right side. The target tooth 101 can move within the moving track of the shell 12 toward the anchor tooth 102 by tensile forces of the elastic member 40. In this situation, the recess 16 is opened nearby the lower canine at the right side and locates below the moving track 122. The extension portion 18 extends downwardly to the center of resistance of the lower molar at the right side. Because the fixing lump 182 is fixed at outer side of the extension portion 18, so tensile forces of the elastic member 40 could not affect the lower molar at the right side. In other possible embodiment, the recess 16 can be opened nearby any target tooth 101 needs to be moved and the extension portion 18 can be positioned accordingly to any fix point nearby the anchor tooth 102.

In other embodiments, for example, the target tooth 101 is an upper canine at the right side or an upper or lower canine at the left side, the recess 16 is opened accordingly nearby the upper canine at the right side, the upper or lower canine at the left side. The anchor tooth 102 related to each said kinds of the target tooth 101 is usually an upper molar at the right side or an upper or lower molar at the left side, the extension portion 18 is positioned accordingly nearby the upper molar at the right side or the upper or lower molar at the left side. However, specifically to illustrate that the target tooth 101 can also be a molar, premolar or even incisor. A person having ordinary skill can easily locate the anchor tooth 102 corresponding to the target tooth 101. For example, the anchor tooth 102 correspondingly is usually a premolar or canine when the target tooth 101 is a molar, the anchor tooth 102 accordingly is usually a molar or canine when the target tooth 101 is a premolar and the anchor tooth 102 accordingly is usually a premolar or canine when the target tooth 101 is an incisor. Based on different the target tooth 101, the recess 16 can be opened accordingly nearby a molar, premolar or canine; meanwhile, the extension portion 18 can be positioned accordingly by a molar, premolar or incisor.

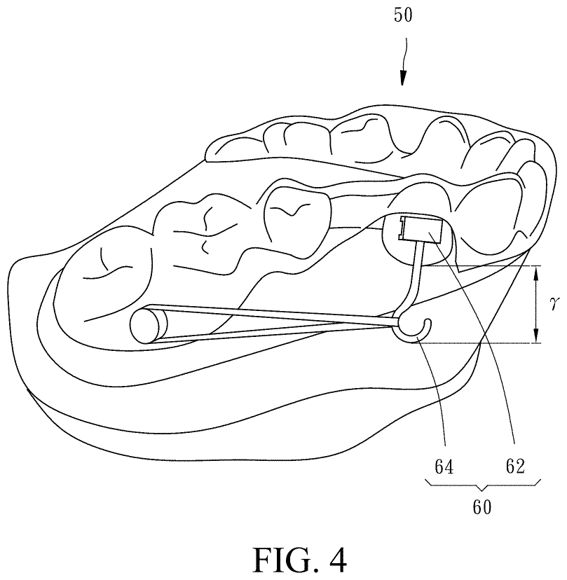

FIG. 4 shows an orthodontic aligner assembly 50 from another embodiment of this invention which is basically the same structure with the orthodontic aligner assembly 20. The only difference is that the orthodontic aligner assembly 50 has a connector 60 having an attaching portion 62 and a hitching portion 64 protruding downwardly from the attaching portion 32 which attach on the outer side of the crown of the target tooth 101. The hitching portion 64 resembles a hook. There is a predetermined distance between the attaching portion 62 and the end of the hitching portion 64 to position it just at the center of resistance of the target tooth 101 when the attaching portion 62 fixed on the crown of the target tooth 101. The distance y between the end of the hitching portion 64 and where the crown of the target tooth 101 and gum meets is about 3 to 5 mm; meanwhile, the distance between the location for the elastic member 40 actually hook on the hitching portion 64 and the end of the hitching portion 64 is no more than 1 mm. This way, the tensile force can equally distribute on the target tooth 101 to move vertically when the hitching portion 64 pulls the target tooth 101 via the contraction of the elastic member 40 and decreases the possibility for the decline of the target tooth 101 during the movement. The connector 60 can be common dental instruments in practice including but not limited to crimpable hook.

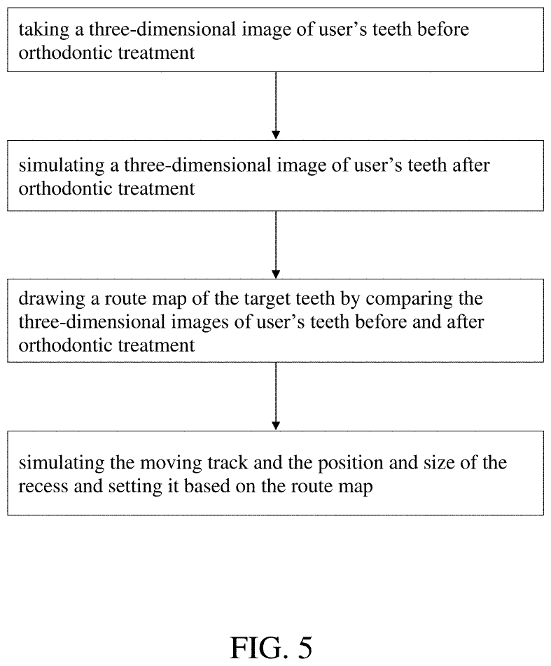

FIG. 5 shows a way for manufacturing the orthodontic aligner 10 of this invention including following steps.

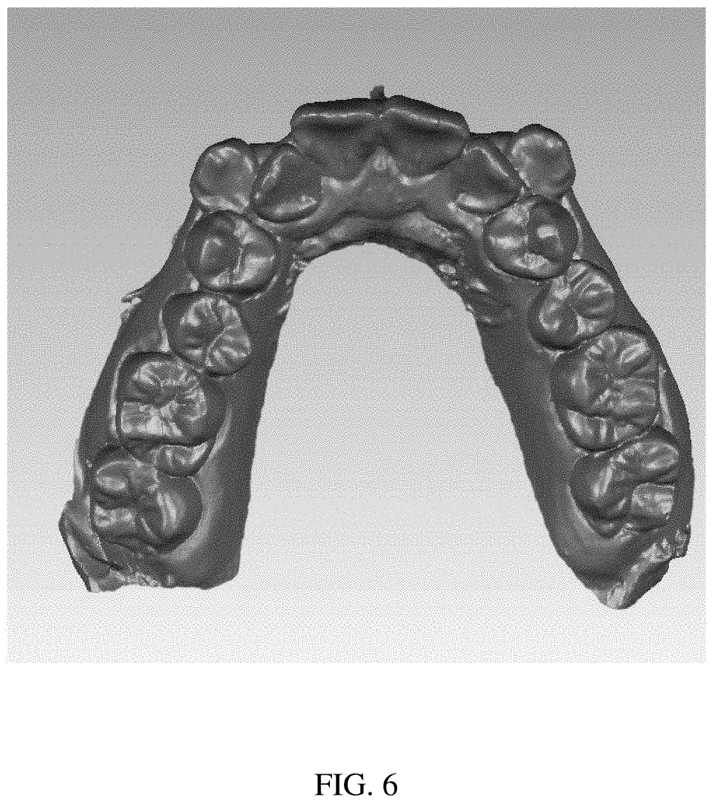

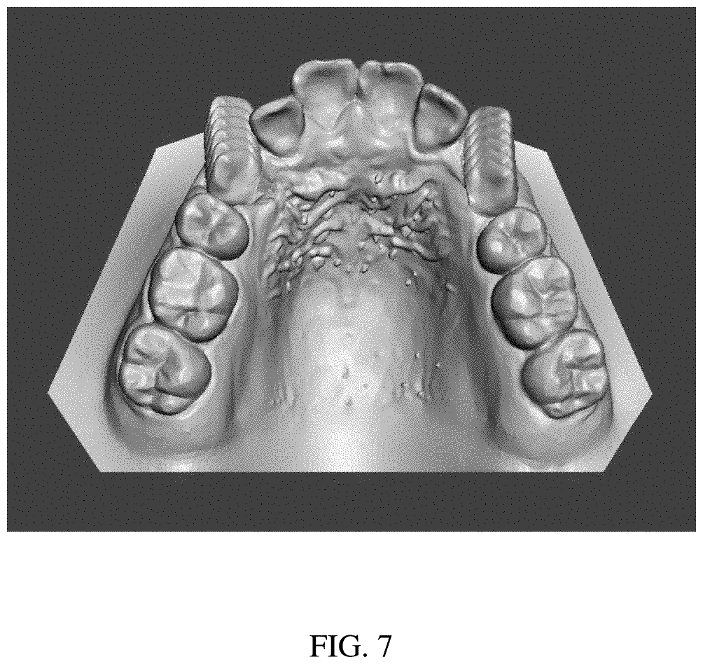

First, a three-dimensional image of user's teeth before orthodontic treatments as illustrated in FIG. 6 is taken. A three-dimensional image of user's teeth after orthodontic treatment is simulated by utilizing a computer software based on the three-dimensional image of user's teeth before orthodontic treatment. A route map of the target teeth 101, a canine for example, is drawn by comparing the three-dimensional images of user's teeth before and after orthodontic treatment. As shown in FIG. 7, this route map is a moving trail combining at least 40 movement points of the target teeth 101. Finally, the moving track 122 and the position and size of the recess 16 are simulated and set based on the route map, and the extension portion 18 and the fixing lump 182 are set based on the fixing location needed when the elastic member 40 pulling the target tooth 101. Wherein, said three-dimensional image of user's teeth, route map, stimulated images or comparing images can be drawn by commercial software such as 3Shape or Inteware.

After said steps, the orthodontic aligner 10 will be placed on user's teeth and the attaching portion 32, 62 of the connector 30, 60 will be fixed on the crown of the target tooth 101.

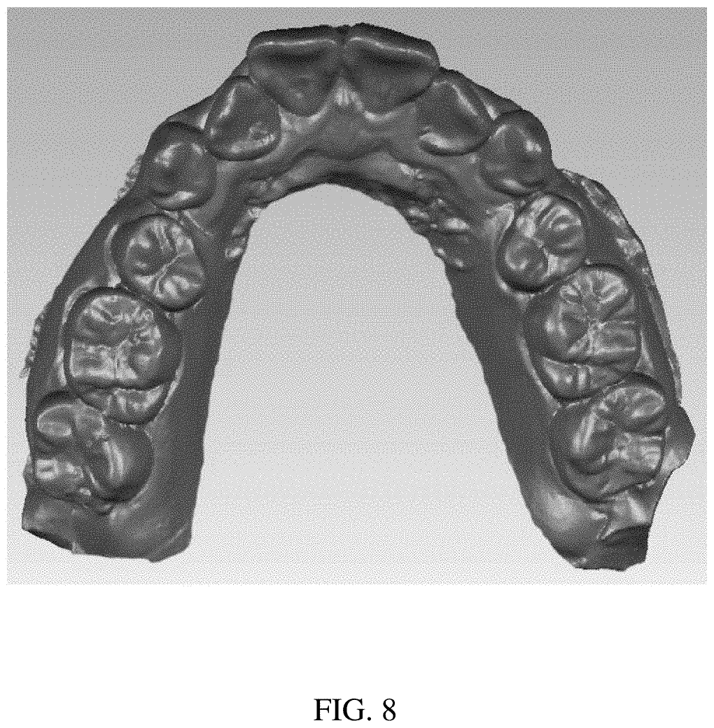

And then, the two ends of the elastic member 40 respectively are wrapped around the fixing lump 182 and the hitching portion 34, 64. The target tooth 101 is then pulled by contracting forces of the elastic member 40 towards the fixing lump 182 on the extension portion 182. By doing so, the space created after a tooth has been removed will be closed. FIGS. 2 and 4 are illustrations of the orthodontic aligner 10 and the orthodontic aligner assembly 20 put together. The three-dimensional image of user's teeth after orthodontic treatment is shown as FIG. 8.

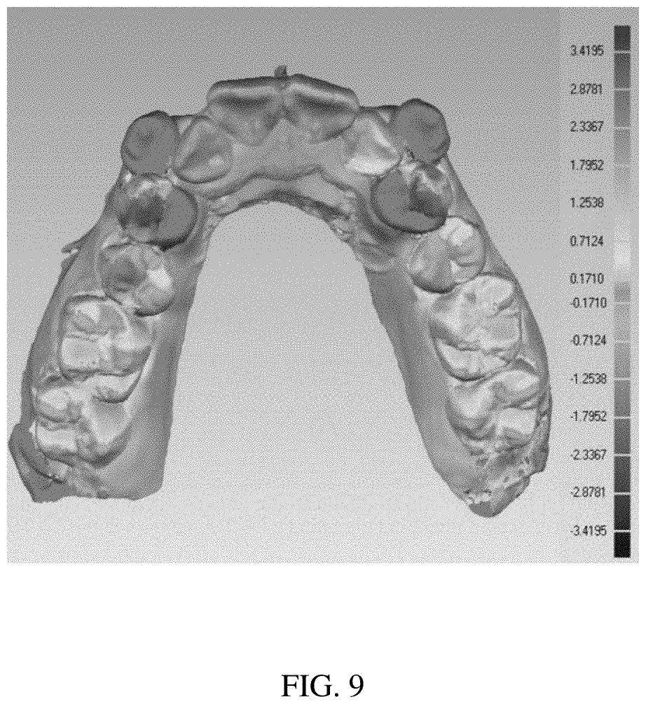

According to above-mentioned structure and technical features, the orthodontic aligner 10 and assembly 20, 50 and, method for manufacturing thereof of this invention is utilized in orthodontic treatment so that the anchor tooth (102) will stay stationary while only the target tooth 101 will be moved during orthodontic treatment. Thus, sequels after orthodontic treatment are reduced, and an invasive temporary anchorage device would not need to be used during orthodontic treatment processes which can further decrease uncomfortableness of user. As FIG. 9 shows, we can see that only the target tooth 101, the gray canine, moved while the other teeth were only displaced±1 mm. By utilizing the recess 16 and the moving track 122 on the orthodontic aligner 10, the target tooth 101 could guide by the moving track 122 and be able to move great distances in just one set and its path will be predictable during orthodontic treatment. Therefore, at least only one of the orthodontic aligner 10 is used to complete the orthodontic treatment, especially for moving one tooth, during whole processes without changing aligner frequently like prior arts to save lots of costs and decrease waste materials.

The above detailed description, which is supported by drawings, is merely intended to provide an embodiment illustrative of the technical content and features of the present invention. The appended claims shall cover simple modifications, replacements or component reduction made, without going against the spirit embodied in the present invention, by persons skilled in the art after gaining insight into the technical content and features of the present invention.

What is claimed is:

1. An orthodontic aligner placed on a user's teeth which include a target tooth and an anchor tooth, the orthodontic aligner comprising:
a shell having a space constructed by the receding of the shell, and a recess constructed through recessing from an edge of the shell at the location corresponding to the target tooth, and an extension portion protruding from the same side of the edge of the shell at the location corresponding to the anchor tooth.

2. The orthodontic aligner of claim 1, wherein the shell is made by one or more of the following materials: PET, PET-G, TPU-PET-G, PE, EVA, PC or PVC.

3. The orthodontic aligner of claim 1, wherein the thickness of the shell is between 0.25 to 0.6 inches.

4. The orthodontic aligner of claim 1, wherein the shell has a wall where the recess formed and a vertical distance between the wall and the top of crown of the target tooth is about 2 to 4 mm.

5. The orthodontic aligner of claim 1, wherein the shell has a wall where the recess formed, and the section around the wall forms a moving track.

6. The orthodontic aligner of claim 1, wherein the fixing lump on the extension portion is located corresponding to the center of resistance of the anchor tooth.

7. An orthodontic aligner assembly, comprising:
an orthodontic aligner of claim 1;
a connector having an attaching portion and a hitching portion protruding from the attaching portion; and
an elastic member with two ends connecting respectively to the fixing lump and the hitching portion.

8. The orthodontic aligner assembly of claim 7, wherein the shell has a wall where the recess formed and a vertical distance between the wall and the central position of the hitching portion is about 2 mm.

9. The orthodontic aligner assembly of claim 7, wherein the attaching portion attaches on the outer side of the crown of the target tooth corresponding to the recess and the hitching portion just at the center of resistance of the target tooth.

10. The orthodontic aligner assembly of claim 7, wherein the distance between the end of the hitching portion and where the crown of the target tooth and gum meets is 3 to 5 mm.

11. A method for manufacturing the orthodontic aligner of claim 1, comprising following steps:
(a) taking a three-dimensional image of user's teeth before orthodontic treatment;
(b) simulating a three-dimensional image of user's teeth after orthodontic treatment;
(c) drawing a route map of the target teeth by comparing the three-dimensional images of user's teeth before and after orthodontic treatment; and
(d) simulating the moving track and the position and size of the recess and setting it based on the route map.

12. A manufacturing method for the orthodontic aligner of claim 11, wherein the route map is a moving trail that combines at least 40 movement points of the target teeth.
